(19) United States
(12) Patent Application Publication
(10) Pub. No.: US 2005/0124978 A1
(43) Pub. Date: Jun. 9, 2005

(54) Indwelling urinary catheter

(76) Inventor: Chul-Jun Kim, Chungcheongnam-do (KR)

Correspondence Address: MCGUIREWOODS, LLP, 1750 TYSONS BLVD, SUITE 1800, MCLEAN, VA, 22102, US

(21) Appl. No.: 10/505,022

(22) PCT Filed: Feb. 21, 2003
(86) PCT No.: PCT/KR03/00359

(51) Int. Cl. A61M 27/00

(52) U.S. Cl. 604/544

(57) ABSTRACT

An indwelling urinary catheter having a simple structure that can discharge urine easily is disclosed. The indwelling urinary catheter comprises a body that includes an inserted part to be inserted into the bladder, and a non-inserted part that is formed in one body with the inserted part and positioned outside of the bladder, a discharging tube installed inside of the body for discharging the urine, the first fixing member formed inside of the inserted part, and an expander one end of which is fixed to the first fixing member. Typical problems associated with the conventional balloon-shaped fixing member are resolved because the indwelling urinary catheter is fixed in a desirable position by an expanding a part of the body. Also, because the indwelling urinary catheter of the present invention needs no additional fixing devices such as the balloon-shaped fixing device but instead can be fixed by the expander, it has a simple structure and enlarged inner diameter of the discharging tube. Thus urine is discharged from the bladder effectively. Moreover, the indwelling urinary catheter has a long life and can be manufactured economically and quickly due to the simple and effective structure.

### TECHNICAL FIELD

The present invention relates to an indwelling urinary catheter, and more particularly to the indwelling urinary catheter that can easily eliminate human urine from a patient's bladder, especially a person who has incontinence as a result of a problem with the sphincter based on a vertebra injury resulting in the bladder to leak urine continuously. In more detail, the present invention overcomes related complications such as the difficulty to insert the catheter into the urethra each time due to the high stiffness of the sphincter or adhesion of the urethra, the severely limited storage capacity of the bladder due to the adhesion of the bladder, and/or the difficulty to utilize the urethral catheterization frequently due to the injury of the urethra.

### BACKGROUND OF THE INVENTION

The urethra catheter for eliminating the urine from the bladder that is most frequently used, is the Foley catheter that has a structure in which a urine inlet hole and fixing device are installed in one side of the body, and urine outlet tube in the center of the body is connected to the urine outlet hole situated in the other side of the body in order to discharge the urine in the patient's bladder.

A urinary catheter based of the above mentioned Foley catheter was disclosed in the Korean patent application No. 2000-22511 entitled ‘the fixing system and method for the urinary catheter’ and Korean patent application No. 2001-29052 entitled ‘Urinary catheter’.

The cross section of the urinary catheter in 2000-29052 is illustrated in the FIG. 1, and FIG. 2 illustrates ‘A’ part in the FIG. 1.

Referring to the FIG. 1 and FIG. 2, an inlet hole 20 through which the urine flows in the catheter, and fixing device 25 to fix the urinary catheter inside of the bladder are installed on the center of one side that is inserted into the bladder. The fixing device 25 has a balloon shape that expands when the liquid for controlling the diameter of the fixing device flows in from the liquid injection device 30 installed in the other side of the urinary catheter. That is, if the liquid for controlling the diameter of the fixing device flows in the fixing device 25 from the liquid injection device 30 via flow tube 35 after inserting one side of the catheter into the bladder, the fixing device 25 expands in the shape of the balloon, and then the urinary catheter become fixed inside of the bladder.

The body of the urinary catheter has a double tube shape, and a urine discharging tube 10 to be connected to the inlet hole 20 is installed at the center of the cross section, and an exudation discharging tube 40 that has plural exudation inlet and outlet holes 45 is installed outside of the urine discharging tube 10. The exudations produced in the urethra flow in the exudation discharging tube 50 through the exudation inlet and outlet holes 45 in the urethra, and then discharged through the exudation inlet and outlet holes 45 outside of the urethra.

Meanwhile, a urine outlet hole 15 to be connected to the urine discharging tube 10 is formed in the other side of the urinary catheter, and urine inside of the bladder is discharged to the outside.

However, although the above mentioned urinary catheter can discharge the urine and exudations simultaneously by the double structure, the urinary catheter can still move from its desired position because the balloon-shaped fixing device is made of soft materials, and even more problematical the overall structure is too complicated because it needs additional devices such as the liquid injection device to expand the balloon. That is, the balloon-shaped fixing device is made of the soft materials for repeated expansion and contraction, so rupturing can easily occur along with shifting from its original position. Moreover there is a danger of foreign particles remaining in the bladder when it ruptures.

Furthermore, there is another danger that the urine might flow backward to the kidneys by excessive pressure caused during the process of injecting the liquid to affix the catheter in the bladder. Also an additional device is required to inject the liquid to expand the balloon, which complicates the production procedures and reduces the inner diameter of the urine discharging tube thus causing decreased effectiveness of the catheter. Finally, the balloon in the end of the urine discharging tube enlarges the outer diameter, which might damage the urethra mucosa and urethra sphincter when inserting the urine discharging tube into the bladder.

An urination controller is also disclosed in Korean Patent Publication No. 97-6099 that prevents the catheter from shifting from its original fixed position. FIG. 3 shows a perspective view of the urination controller, and FIG. 4 shows a cross section illustrating when the urination controller is inserted into the bladder in FIG. 3.

Referring to the FIG. 3 and FIG. 4, the urination controller includes the urine discharging tube 65 connected to the valve 60 at one side. An external outlet 70 is formed in one end, and plural sidewall openings 75 are formed at the other end.

An air tube 105 extends facing to the inlet 80 to the inside of the valve 60 and is arranged along to the longitudinal direction of the urine discharging tube 65. At the both ends of the air tube 105, an outlet 90 positioned inside of the device 85 forming a part of the sidewall of the urine discharging tube 65, and inlet 115 are formed. If the air is injected to the device 85 on the sidewall of the urine discharging tube 65 via the air tube 105, the device 85 expands to form a balloon shaped chamber 100. To make this possible, an opening 120 is formed on one side of the air tube 105.

To prevent the urination controller from shifting from its fixed position, a fixing collar 95 is formed outside of the bladder corresponding to the chamber 100 inside of the bladder. Thus, the urination controller can remain in its original fixed position due to both the chamber 100 inside of the bladder and the fixing collar 95 outside of the bladder.

However, because the urination controller is affixed by inserting air into the balloon-shaped chamber made of the soft material, additional devices are required such as the air tube for inserting air, which still leaves the same problem when a rupture of the balloon-shaped chamber occurs.

### SUMMARY OF THE INVENTION

Therefore, it is an object of the present invention to provide an indwelling urinary catheter that can be easily fixed inside of the bladder of the patient without the balloon shaped fixing device by making the body of the indwelling urinary catheter expand partially.

To accomplish the objects of the present invention, there is provided a indwelling urinary catheter including an inserted part to be inserted into the bladder, a body that includes the inserted part and non-inserted part is formed in one body with the inserted part and exposed to the outside of the bladder of the patient, a discharging tube installed inside of the body to discharge the urine, the first fixing member formed inside of the inserted part, and an expander fixed to the first fixing member at its one end. In this case, the first fixing member has the opened-ring or closed-ring shape, and plural inlet holes are formed in its circumference of one end through which the urine flows in from the bladder.

Preferably the plural openings are shaped like a running track, lozenge, oval or polygon that can be formed in the inserted part. Accordingly, the expander can be formed from wire selected from steel wire, piano wire, or typical fishing-type line.

According to one aspect of the present invention, a part of the inserted part expands in the lateral direction of the body with the opening as the center by pulling the free end of the expander, and then the indwelling urinary catheter is fixed inside of the bladder of the patient. An indicator that is an area or points having a different color from the rest of the expander, can be included to indicate the expanding state of the inserted part.

Also, an inlet hole can be formed in one end of the discharging tube positioned inside of the body, and an outlet hole can be formed in the other end of the discharging tube positioned outside of the body. The second fixing member that consists of a protrusion, a recess, or a combination of the protrusion and the recess is formed at one end of the discharging tube to fix the one end of the expander.

According to another aspect of the present invention, a contacting member is formed next to the openings of the inserted part, and a ring shaped mounting recess is formed between the inserted part and non-inserted part.

According to still another aspect of the present invention, a central hole through which the expander passes is formed in the center of one end of the discharging tube positioned inside of the body, and the end has a cone shape, in the circumference of which plural inlet holes are formed, and the outlet hole is formed on other end of the discharging tube.

Also, the first thread is formed on the inner face of the non-inserted part, the second thread corresponding to the first thread is formed on the outer face of the discharging tube, and the second fixing member which is one among protrusion, a recess or combination of the protrusion and recess, is formed in the other side of the discharging tube on which the other side of the expander is rolled and fixed. Thus, as the discharging tube is withdrawn to the outside by rotation, the inserted part expands partially in the lateral direction of the body with the opening as the center. In this case, an indicator is formed on the outer face of the other side of the discharging tube, by which the expanding state of the inserted part can be indicated.

Also, according to still another aspect of the present invention, a indwelling urinary catheter is provided that includes a body comprising an inserted part for being inserted into the bladder and non-inserted part for being positioned outside of the bladder, a expanding part between the inserted part and the non-inserted part, a discharging tube installed inside of the body and discharging the urine, a fixing member formed inside of the one end of the inserted part and an expander fixed to the fixing member at one side.

Preferably, the expanding part has a ring shape, on the outer face of which plural openings are formed, and to prevent the expanding part from being separated, the first protrusion that is inserted into the one end of the expanding part is formed on one end of the inserted part and the second protrusion that is inserted into the other end of the expanding part is formed on one end of the non-inserted part.

According to the present invention, because a part of the body expands to fix the indwelling urinary catheter inside of the bladder of the patient, typical problems associated with the conventional balloon shaped fixing device can be solved. Also, because the indwelling urinary catheter can be easily affixed by the expander without additional devices such as air pump or liquid pump, the overall structure is simple and the urine in the bladder can be effectively discharged by enlarging the inner diameter of the discharging tube. Moreover, the indwelling urinary catheter has a longer life and can be manufactured inexpensively and quickly due to the simple and effective structure.

### BRIEF DESCRIPTION OF THE DRAWINGS

The above objects and other advantages of the present invention will become more apparent by detailed describing preferred embodiments thereof with reference to the attached drawings in which:

FIG. 1 is the cross sectional view of a conventional urethra catheter;

FIG. 2 is the cross sectional view of the part ‘A’ in the Fig.;

FIG. 3 is the perspective view of the conventional urination controller;

FIG. 4 is the cross sectional view of the urination controller in FIG. 3 to illustrate the state that the urination controller is inserted into the bladder;

FIG. 5 is the perspective view of the first embodiment of the present invention;

FIG. 6 is the cross sectional view according to the line B1-B2 in FIG. 5;

FIG. 7 is the partially projected view of the first embodiment of the present invention to show the operation of the indwelling urinary catheter;

FIG. 8 is the plane view of the expander in the first embodiment of the present invention;

FIG. 9 is the perspective view of the second embodiment of the present invention;

FIG. 10 is the enlarged cross sectional view of the contacting member in FIG. 9;

FIG. 11 is the perspective view of the third embodiment of the present invention;

FIG. 12 is the cross sectional view according to the line D1-D2 in FIG. 11;

FIG. 13 is the front view of the discharging tube in the FIG. 12;

FIG. 14 is the perspective view of the fourth embodiment of the present invention;

FIG. 15 is the cross sectional view according to the line E1-E2 in FIG. 14;

FIG. 16 is the perspective view of the fifth embodiment of the present invention;

FIG. 17 is the cross sectional view according to the line F1-F2 in FIG. 16;

FIG. 18 is the partially projected view of the fifth embodiment of the present invention to show the operation of the indwelling urinary catheter;

FIG. 19 is the perspective view of the sixth embodiment of the present invention;

FIG. 20 is the cross sectional view according to the line G1-G2 in FIG. 19;

FIG. 21 is the partially projected view of the sixth embodiment of the present invention to show the operation of the indwelling urinary catheter;

FIG. 22 is the side view of the sixth embodiment of the present invention in FIG. 19; and

FIG. 23 is the partially cut cross sectional view of the one end of the non-inserted part 720.

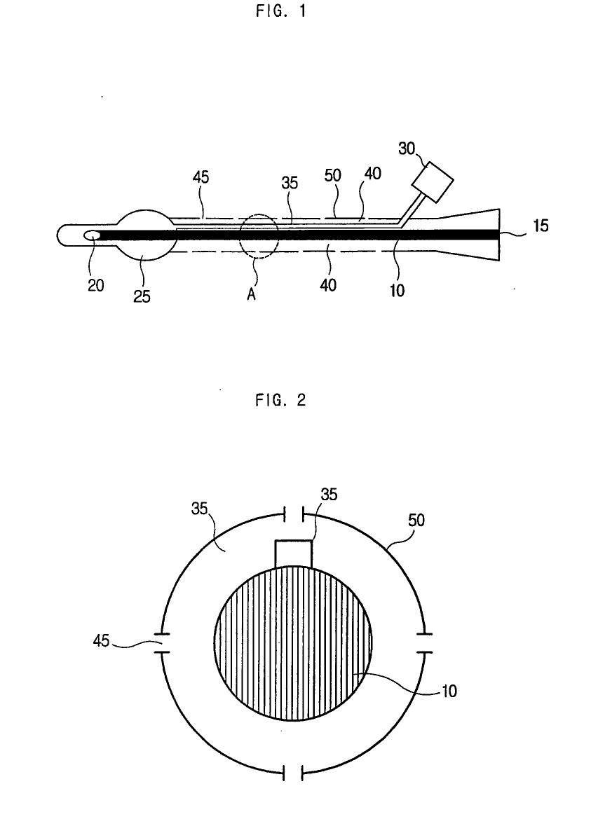

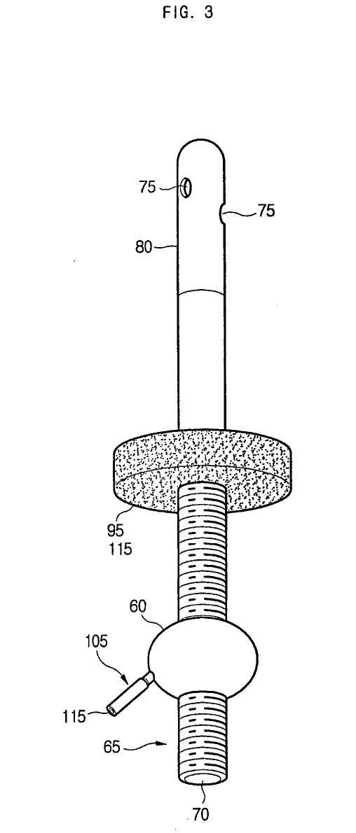

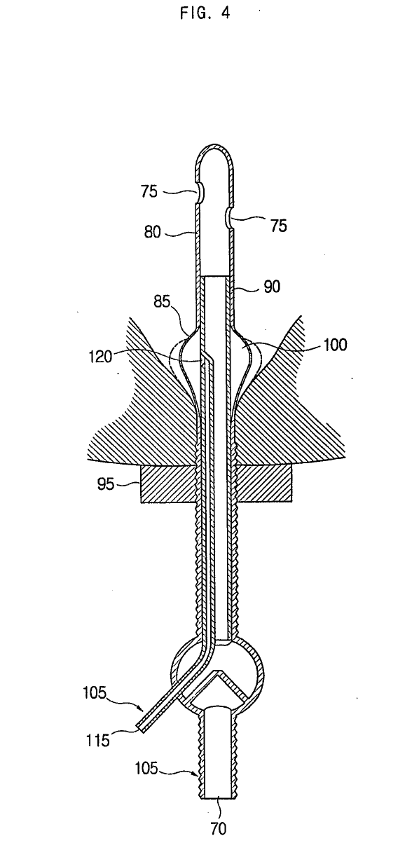

### EMBODIMENTS

Hereinafter, preferred embodiments of the present invention will be described in more detail with reference to the accompanying drawings, but it is understood that the present invention should not be limited to the following embodiments.

### Embodiment 1

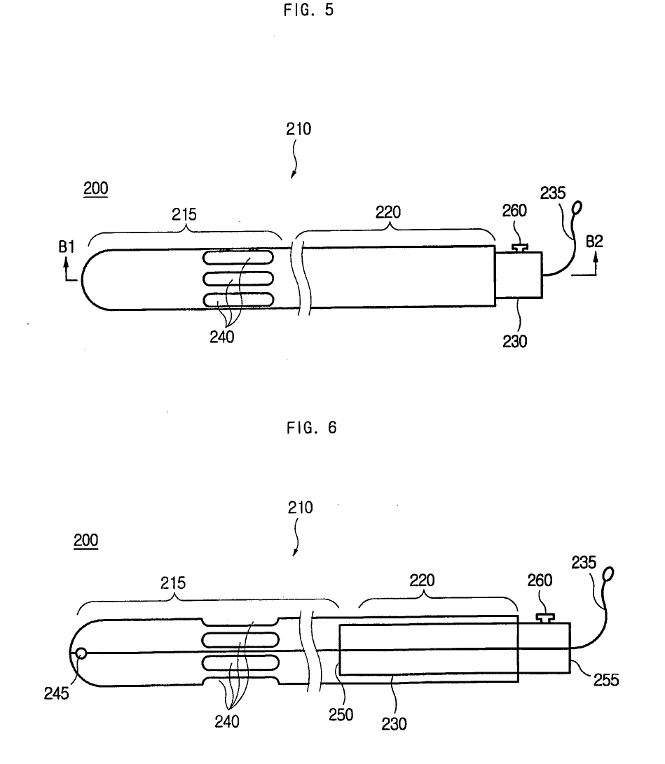

FIG. 5 is the perspective view of the first embodiment of the present invention, and FIG. 6 is the cross sectional view according to the line B1-B2 in FIG. 5.

Referring to the FIG. 5 and FIG. 6, the indwelling urinary catheter 200 of the first embodiment includes an inserted part 215 to be inserted into the bladder, a body 210 that includes the inserted part 215 and non-inserted part 220 is formed in one body with the inserted part and exposed to the outside of the bladder of the patient, a discharging tube 230 installed inside of the body 210 to discharge the urine, and an expander 235 fixed to the one end of the body 210 and penetrating the discharging tube 230.

The inner end of the inserted part 215 that forms one side of the body 210 has the first fixing member 245 to affix the expander 235. To the first fixing member 245 the one end of the expander 235 is connected, and the other end of the expander 235 is exposed to the outside of the outlet hole 255 through the discharging tube 203. The first fixing member 245 can have the shape of an opened ring or closed ring for easy connection to the one end of the expander 235.

The inserted part 215 can have a pre-determined radius at its one end for easy insertion, and plural openings 240 that are track shaped holes are formed in the inserted part along to the circumference of the body with same interval and size in the longitudinal direction of the body in the adjacent area to the non-inserted part. For the extraction and contraction of the body 210, the body 210 is made of the synthetic rubber, silicon resin or polyurethane resin. Also, the body 210 can be made of the synthetic rubber that is manufactured by applying the N-halamine to the polystyrene to eliminate the pathogen such as the virus or mycota. This alternative structure is very useful to a patient who has weak immunity due to “AIDS”, cancer, or organ transplantation.

As described above, by forming plural openings 240 in a part of the body 210, and upon pulling the free end of the expander 235 connected to the first fixing member 245, a longitudinal force is generated in the body 210 of the indwelling urinary catheter 200, and then the part in which the openings 240 are formed expands laterally by the force. This operation will be discussed later.

And, the openings 240 perform the same function as the inlet holes of the conventional urethra catheter. Thus, the indwelling urinary catheter of the present invention 200 can discharge the urine in the bladder of the patient through the openings 240 without forming additional inlets.

Moreover, because the indwelling urinary catheter of the present invention 200 needs no additional fixing devices such as the balloon shaped fixing device, a simple structure will suffice and the inner diameter of the discharging tube 230 of the body 210 is enlarged. Thus urine in the bladder of the patient is discharged in a very short time. To assist such function, plural inlet holes can be formed in one end of the inserted part 215 of the body 210 like the conventional catheter.

The non-inserted part 220 of the body 210 is the part that will be positioned outside of the bladder of the patient, when the inserted part 215 is inserted into the bladder of the patient and the openings 240 expands. The discharging tube 230 is installed in the non-inserted part 220 that has an inlet hole 250 in one end through which the urine flows in from the bladder, and an outlet hole 255 in the other end through which the urine is discharged to the outside. The one end of the discharging tube 230 extends beyond the non-inserted part 220 and is arranged adjacent to the openings 240 of the inserted part 215, and the other end of the discharging tube 230 extends to the outside of the non-inserted part 220 of the body.

The expander 235 made of a wire such as the piano wire, steel wire or fishing-type line is connected to the first fixing member 245 formed in the inserted part 215 in its one end, and the other end is exposed to the outside through the discharging tube 230.

In the same way, the second fixing member 260 is formed on the outer face of the end of the exposed discharging tube 230 on which the other end of the expander 235 is rolled and fixed. In FIG. 5 and FIG. 6, the second fixing member 260 is illustrated as the protrusion shape, but it can be formed as a recess. And the end of the expander 235 can be fixed more tightly by forming the protrusion and recess together. The second fixing member 260 maintains the body 210 in the expanded state by rolling the end of the expander 235 on the second fixing member 260 after expanding the part in which the openings 240 are formed among the body 210 by pulling the end of the expander 235.

In the first embodiment, the openings are formed like an extended track shape, but they can also be formed in a different shape such as an extended lozenge, an oval, or a rectangle according to the optimal expanding height. In particular, when the openings have the lozenge shape that is extended to the non-inserted part, leakage of the urine is effectively prevented from the bladder due to the increased degree of contact because the expanding height of the non-inserted part become higher than that of the inserted part.

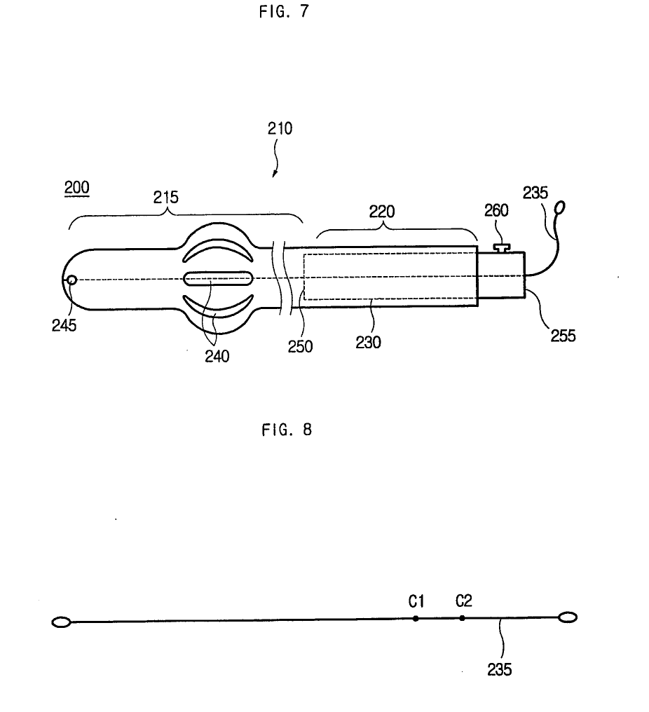

Hereafter, the operations of the first embodiment will be described referring to the FIG. 7.

FIG. 7 is the partially projected view of the first embodiment of the present invention to show the operation of the indwelling urinary catheter.

Referring to the FIG. 7, by pulling the exposed end of the expander 235 after inserting the inserted part 215 of the body 210 into the bladder of the patient, a part of the inserted part 215 folds along to the lateral direction of the body 210 with the openings 240 formed in the extended track shape in the longitudinal direction of the body as a center, and then the indwelling urinary catheter is fixed in the bladder of the patient. In this case, the urine in the bladder of the patient flows in the discharging tube 230 through the plural openings 240 formed in the inserted part 215 and inlet hole 250 of the discharging tube 230, and thereafter is discharged to the outside through the outlet hole 255 of the discharging tube 230. Forming additional openings on the outer face of the inserted part 215 can accelerate the discharging of the urine.

Therefore, because the indwelling urinary catheter of the present invention can be fixed in the bladder of the patient by the partial expanding of its body 210 without additional fixing device, the problem with a conventional urethra catheter using a balloon-shaped fixing device that is a required additional device and has the potential to rupture resulting in further health complications can be resolved.

FIG. 8 is the plane view of the expander 235 in the first embodiment of the present invention. As shown in FIG. 8, the expander 235 can include an indicator located in the range of C1˜C2. The indicator has a different color in contrast to the entire color of the expander 235; for example, the indicator has a yellow color whereas the entire expander 235 has a black color, or the indicator has a red or blue color whereas the entire expander 235 has a white color.

The indicator indicates the degree to which a part of the body 210 expands when partially expanding the body 210 by pulling the expander 235. It is difficult to know the degree to which a part of the body 210 expands outside, because the inserted part 215 expands inside of the bladder of patient with the openings 240 as a center. Thus, by using the expander 235 having the indicator, whether the part of the inserted part 215 expands optimally can be readily checked.

In FIG. 8, the indicator is an area in the range of C1-C2, but the indicator can be the points arranged in the range of C1˜C2 with a different color to the entire expander 235.

### Embodiment 2

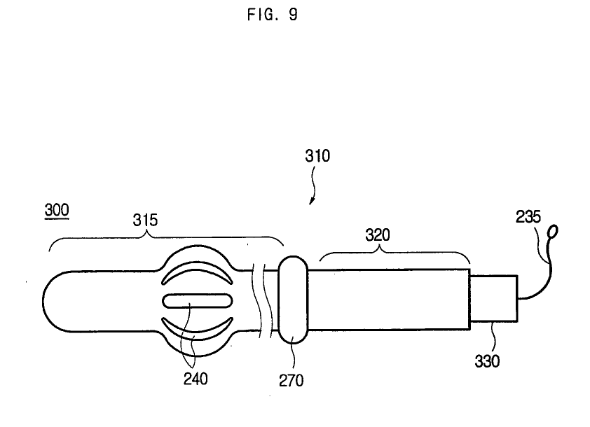

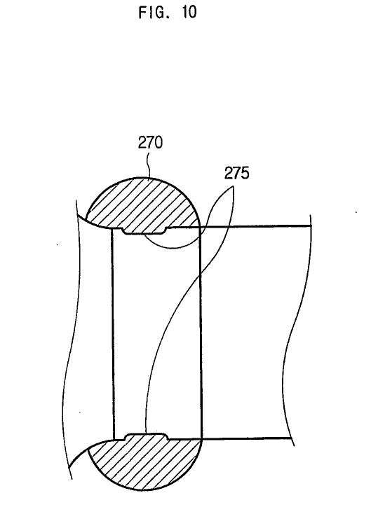

FIG. 9 is the perspective view of the second embodiment of the present invention, and FIG. 10 is the enlarged cross sectional view of the contacting member in FIG. 9.

Referring to the FIG. 9 and FIG. 10, the indwelling urinary catheter 300 in the second embodiment has same structure as in the first embodiment except that a contacting member 270 having a ring shape is further included in the inserted part 315 next to the openings 340 of the body 310.

The contacting member 270 is made of elastic material such as silicon rubber, supports the expanded part when a part of the inserted part 315 expands in the lateral direction of the body 310, and prevents leakage of the urine between the expanded part and bladder. To fix the contacting member 270 to the body 310, a ring-shaped recess 275 is formed in the outer circumference of the body 310 between the inserted part 315 and the non-inserted part 320.

Thus, the indwelling urinary catheter 300 in the second embodiment can maintain partial expansion of the inserted part 315 by the expander 335, and still prevent leakage of the urine from the bladder of the patient.

### Embodiment 3

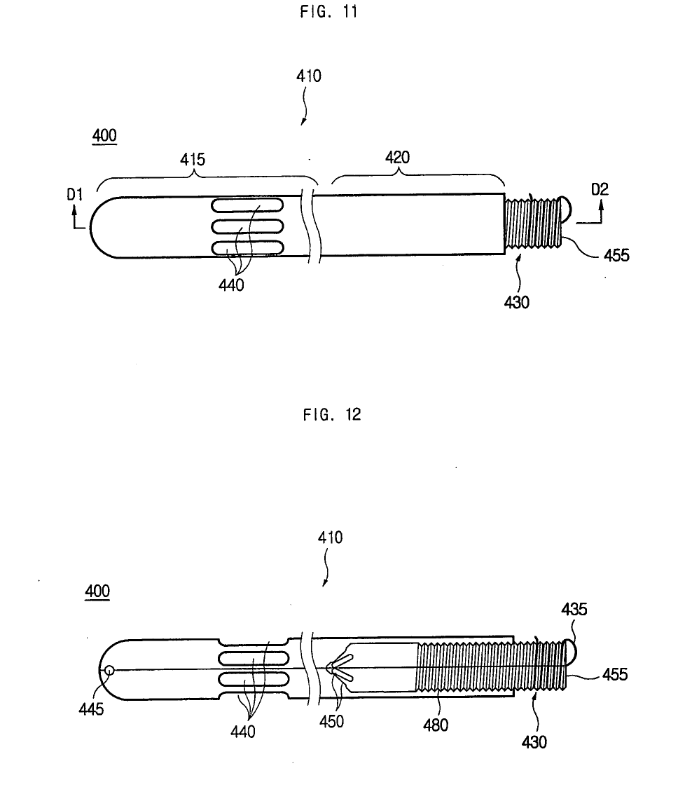

FIG. 11 is the perspective view of the third embodiment of the present invention, and FIG. 12 is the cross sectional view according to the line D1-D2 in FIG. 11.

Referring to the FIG. 11 and FIG. 12, the indwelling urinary catheter 400 in the third embodiment includes a body 410 comprising an inserted part 415 and non-inserted part 420, a discharging tube 430 installed in the body 410, and an expander 435 to expands a part of the body 410. Because it is the same as in the first embodiment that the first fixing member 445 is formed inside of the one end of the inserted part 415 and plural openings 440 are formed in outer face of the inserted part 415, the detailed description will be omitted.

Plural inlet holes 450 are formed in one end of the discharging tube 430, and the outlet hole 455 to discharge the urine in the bladder of the patient is formed in the other end of the discharging tube 430. The part of the discharging tube 430 in which the inlet holes 450 are formed is in a cone shape, and center hole 485 through which the expander 435 penetrate is formed in the center of the cone.

Also, the first thread 480 is formed on the inner face of the non-inserted part 420 to be positioned outside of the bladder of the patient, and the second thread 470 corresponding to the first thread 480 is formed on the outer face of the discharging tube 430 that is the path of the urine to be discharged. Thus, if rotating the discharging tube 430 with the second thread 470, the second thread 470 of the discharging tube 430 will rotate engaged with the first thread 480 of the non-inserted part 420, and then the discharging tube 430 will be withdrawn to the outside of the non-inserted part 420. As the discharging tube 430 is withdrawn to the outside of the non-inserted part 420, the longitudinal force will be exerted on the inserted part 415 by the expander 435 that is fixed to the first fixing member 445 in one end, and then the part in which the openings 440 are formed will expand in the longitudinal direction. In this situation, the second fixing member can be formed in one end of the discharging tube 430 and be one among a protrusion, recess or a combination of the protrusion and recess.

To check whether the inserted part 415 expanded optimally, an indicator can be included in a predetermined position of the expander 435 or one end of the discharging tube 430 that is in the non-inserted part 420. That is, to check whether the discharging tube exited optimally beyond the body 410, the indicator comprising an area or points that has a contrasting color with the color of the entire body can be included in the one end of the discharging tube 430.

FIG. 13 is the front view of the discharging tube in the FIG. 12. As shown in the FIG. 13, the one end of the discharging tube 430 is formed in a cone shape, the center hole 485 is formed in the center of the cone, and plural inlet holes 450 are formed in the circumference of the center. The expander 435 penetrates through the center hole 485 to be fixed to the first fixing member 445, and the urine flows into the discharging tube 430 through the inlet holes 450. When the discharging tube 430 is withdrawn outside of the body 410 via rotation, the expander 435 will pull the inserted part 415 out accurately in a direction corresponding to the lateral direction of the body 410, and then the part in which the openings 440 are formed can expand in the longitudinal direction optimally.

### Embodiment 4

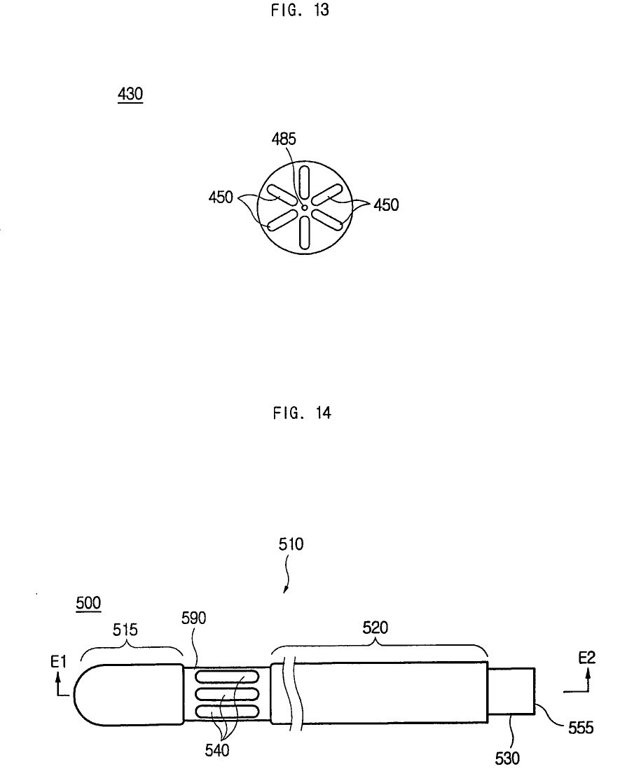

FIG. 14 is the perspective view of the fourth embodiment of the present invention, and FIG. 15 is the cross sectional view according to the line E1-E2 in FIG. 14.

Since the fourth embodiment has the same structure as in the above-described embodiments except that the expanding part is installed between the inserted part and non-inserted part, the description for the same structure will be omitted.

Referring to the FIG. 14 and FIG. 15, the indwelling urinary catheter 500 of the fourth embodiment includes a body 510 that comprises an inserted part 515 and non-inserted part 520, a discharging tube 530 positioned in the body 510, and a ring shaped expanding part 590 formed between the inserted part 515 and non-inserted part 520.

The expanding part 590 with plural openings 540 is made of the soft and elastic material such as the silicon rubber, and installed between the inserted part 515 and non-inserted part 520 of the body 510. As shown in the FIG. 6 and FIG. 12, the indwelling urinary catheter 500 of the fourth embodiment can also include the same expander as in the first embodiment to expand the expanding part 590 in the longitudinal direction of the body 510, and the threads in the discharging tube 530 and non-inserted part 520. Since the expander and threads were fully described above, their detailed description will be omitted.

To prevent the expanding part from separating while fixing the indwelling urinary catheter 500 of the fourth embodiment by expanding the expanding part after inserting into the bladder of the patient, the first and second protrusions 595, 596 are formed in the inserted part 515 and non-inserted part 520 respectively. The first and second protrusions 595, 596 extend into the expanding part 590 individually to prevent the expanding part from separating from the body 510. Also, the indwelling urinary catheter 500 of the fourth embodiment can include plural inlet holes on the outer face of the inserted part. 515 to effectively discharge the urine of the bladder through the discharging tube 530 via inlet hole 550 and outlet hole 555 at each end.

### Embodiment 5

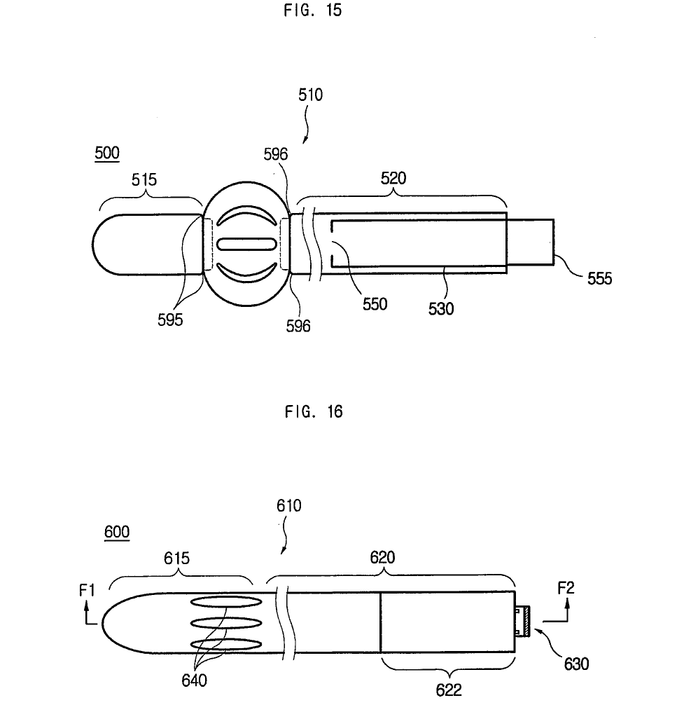

FIG. 16 is the perspective view of the fifth embodiment of the present invention, and FIG. 17 is the cross sectional view according to the line F1-F2 in FIG. 16.

As shown in the FIG. 16 and FIG. 17, the indwelling urinary catheter 600 of the fifth embodiment includes a body 610 comprising an inserted part 615 and a non-inserted part 620, a discharging tube 630 positioned in the body 610, and an expander 635 to expand a part of the body 610. Since the first fixing member 645 and the inserted part 615 with plural openings 640 in the circumference are the same as in the first embodiment, their detailed description will be omitted.

Plural inlet holes 650 are formed in one end of the discharging tube 630, and the outlet hole 655 is formed in the other end of the discharging tube 630 to discharge the urine from the bladder. The part of the discharging tube 630 in which the inlet holes 650 are formed has a cone shape, and the expander 635 is fixed in the center of the cone. Also a contacting member 660 is installed next to the inlet holes 650. To easily insert and withdraw the discharging tube 630 assembled in the contacting member 660, preferably the contacting member 660 is made of material such as plastic that has certain degree of rigidity.

An external fixing member 670 is installed in the other end of the discharging tube 630. The one end of the external fixing member 670 is fixed to one end of the discharging tube 630, and the other end of the external fixing member 670 is received in the non-inserted part 620. That is, the one end of the external fixing member 670 operates as a hinge, and the free end of the external fixing member 670 can be bent towards the discharging tube 630 to be inserted into the non-inserted part 630. If the discharging tube 630 is withdrawn to the outside, the free end of the external fixing member 670 will return to its original position parallel to the outer face of the discharging tube 630 or bent toward outside to engage in the recess 625 in the end of the non-inserted part 620.

Also, a membrane 680 to prevent leakage of the urine is installed in one end of the discharging tube 630. By using the membrane 680, typical leakage of urine that occurs while inserting the indwelling urinary catheter can be effectively prevented. If the indwelling urinary catheter 600 is inserted into the bladder, the urine that flows into the discharging tube 630 through the inlet holes 650 will be discharged through the outlet hole 655. Meanwhile the membrane 680 closes the outlet hole, and then a urine collecting tube 800 is inserted into the discharging tube 630 through the membrane 680. Thus, preliminary action such as closing the discharging tube using a clamp before insertion into the bladder is not necessary.

As the discharging tube 630 is withdrawn to the outside of the non-inserted part 620 of the body 610, the longitudinal force will be exerted on the inserted part 615 by the expander 635 that is fixed to the first fixing member 445 in one end, and then the part in which the openings 640 are formed will expand in the longitudinal direction of the body 610.

Hereafter, the operation state will be described with reference to the drawings.

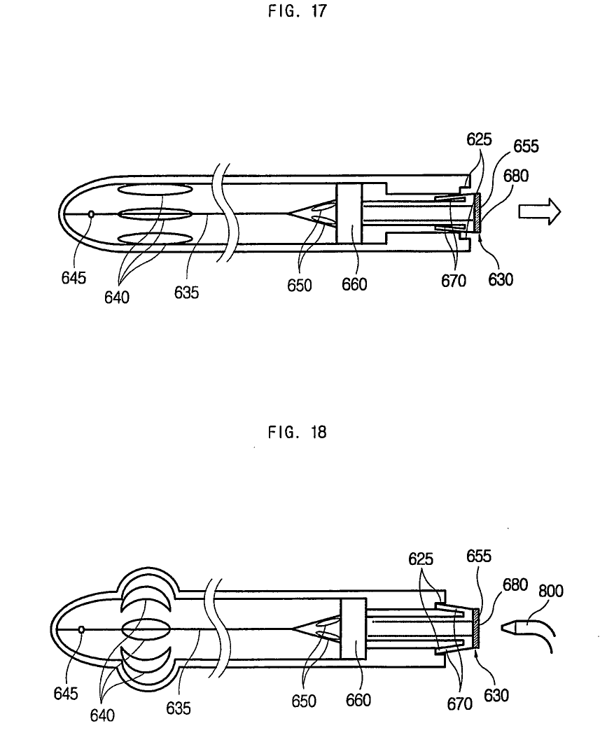

FIG. 18 is the partially projected view of the fifth embodiment of the present invention to show the operation of the indwelling urinary catheter.

Referring to the FIG. 18, by pulling the one end of the discharging tube 630 after inserting the inserted part 615 of the body 610 of the indwelling urinary catheter into the bladder of the patient, a part of the inserted part 615 folds along to the lateral direction of the body 610 with the openings 640 forming an extended track shape in the longitudinal direction of the body 610 as a center, and then the indwelling urinary catheter 600 is fixed in the bladder of the patient. In this case, the urine in the bladder of the patient flows in the discharging tube 630 through the plural openings 640 formed in the inserted part 615 and inlet hole 650 of the discharging tube 630, and then discharges outside through the outlet hole 655 of the discharging tube 630. Forming additional openings on the outer face of the inserted part 615 can accelerate the discharging of the urine. The discharging tube 630 the one end of which is withdrawn to the outside maintain the indwelling urinary catheter 600 fixed by engaging the external fixing member 670 in the recess 625.

When the indwelling urinary catheter 600 is fixed, the urine discharged from the bladder is collected by connecting the urine collecting tube 800 to the outlet hole 655. The urine collecting tube 800 is inserted into the outlet hole 655 penetrating through the membrane 800 to be fixed in the indwelling urinary catheter 600.

### Embodiment 6

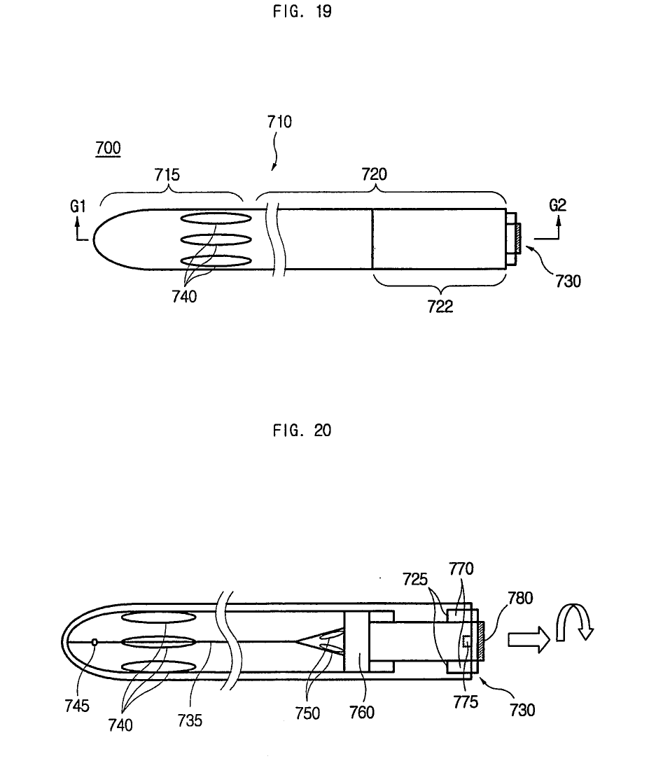

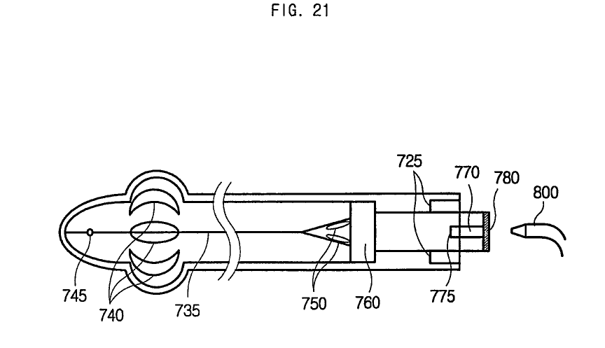

FIG. 19 is the perspective view of the sixth embodiment of the present invention, and FIG. 20 is the cross sectional view according to the line G1-G2 in FIG. 19.

Since the indwelling urinary catheter in the sixth embodiment has the same structure as in the fifth embodiment except that a fixing protrusion is formed in one end of the discharging tube and the first and second recess are formed in the one end of the non-inserted part, their detailed description will be omitted.

Referring to the FIG. 19 and FIG. 20, the indwelling urinary catheter 700 in the sixth embodiment includes a body 710 comprising an inserted part 715 and a non-inserted part 720, a discharging tube 730 positioned in the body 710, and an expander 735 to expand a part of the body 710. To easily insert and withdraw the discharging tube 730 assembled in the contacting member 760, preferably, the contacting member 760 is made of the material such as the plastic that has certain degree of rigidity. Also, since the contacting member 760 is pressed to a certain degree while fixing the discharging tube 730 in the second fixing recess 775 by withdrawing the discharging tube 730 to fix the indwelling urinary catheter 700, preferably, the contacting member is made of elastic material.

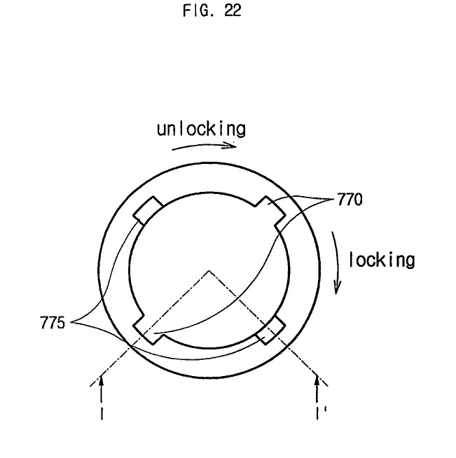

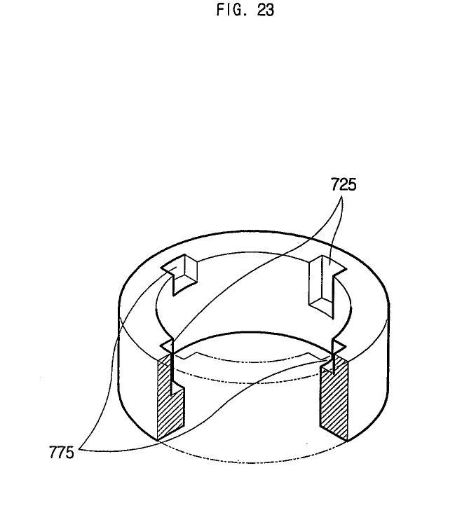

At least one protrusion 770 that is an external fixing member is formed on the outer face of the end of the discharging tube 730. If plural protrusions are formed, the angle between each protrusion is preferably the same for all the protrusions. The protrusion, the first recess, and second recess will be described with reference to the FIG. 22 and FIG. 23. FIG. 22 is the side view of the sixth embodiment of the present invention in FIG. 19, and FIG. 23 is the partially cut cross-sectional view of the one end of the non-inserted part 720.

If the discharging tube 730 is received in the non-inserted part 720, the protrusions 770 are positioned in the first recess 725. The first recesses 725 are formed on the inner face of the end of the non-inserted part 720, the number of the first recesses 725 are the same as the number of the protrusions 770, and it is preferred to form two protrusions 770 symmetrically. Also, it is preferred to form the first recesses 725 in such a depth as a part of the inserted part 715 that cannot expand in the longitudinal direction of the body 710 by the expander 735.

If the discharging tube 730 is withdrawn to the outside of the non-inserted part 720, the protrusions 770 are positioned in the second recesses 775. The second recesses 775 are formed on the inner face of the end of the non-inserted part 720, the number of the second recesses 775 are the same as the number of the protrusions 770, and it is preferred to form two protrusions 770 symmetrically. Also, it is preferred to form the second recesses 775 in such a depth as a part of the inserted part 715 that cannot expand in the longitudinal direction of the body 710 by the expander 735.

And, it is also preferred to form the first recessed 725 deeper than the second recessed 775, so that the part in which the openings 740 are formed will remain in an expanded state in the longitudinal direction of the body 710 by the discharging tube 730 with the protrusions fixed to the second recessed 775.

Hereafter, the operation state will be described with reference to the drawings.

FIG. 21 is the partially projected view of the sixth embodiment of the present invention to show the operation of the indwelling urinary catheter.

Referring to the FIG. 21, by pulling the one end of the discharging tube 730 after inserting the inserted part 715 of the body 710 of the indwelling urinary catheter into the bladder of the patient, a part of the inserted part 715 folds along to the longitudinal direction of the body 710 with the openings 740 forming an extended track shape in the longitudinal direction of the body 710 as a center, and then the indwelling urinary catheter 700 is fixed in the bladder of the patient. In this situation, the urine in the bladder of the patient flows in the discharging tube 730 through the plural openings 740 formed in the inserted part 715 and inlet hole 750 of the discharging tube 730, and then discharges outside through the outlet hole 755 of the discharging tube 730. Forming additional openings on the outer face of the inserted part 715 can accelerate the discharging of the urine. The discharging tube 730 one end of which is exposed to the outside maintains the indwelling urinary catheter 700 in a fixed position by engaging the protrusions 770 in the second recesses 775 due to the clockwise or counter clockwise rotation of the discharging tube 730.

When the indwelling urinary catheter 700 is fixed, the urine discharged from the bladder is collected by connecting the urine collecting tube 800 to the outlet hole. The urine collecting tube 800 is inserted into the outlet hole penetrating through the membrane 800 to be fixed in the indwelling urinary catheter 700.

In the above embodiments, the openings are formed having an extended track shape, but they can be also formed in the different shape such as an extended lozenge, oval, or rectangle according to the optimal expanding height. In particular, when the openings have the lozenge shape that is extended to the non-inserted part, there is no need to include an additional contacting member to prevent leakage of the urine due to the increased degree of contact because the expanding height of the non-inserted part becomes higher than that the inserted part.

### INDUSTRIAL UTILIZABILITY

According to the present invention, problems with the conventional balloon shaped fixing member can be resolved because the indwelling urinary catheter can be fixed in a desirable position by expanding a part of the body. Furthermore, the indwelling urinary catheter of the present invention needs no additional fixing devices such as the balloon-shaped fixing device but instead can be fixed by the expander. Also the indwelling urinary catheter has a simple structure and enlarges the inner diameter of the discharging tube, thus discharging the urine in the bladder effectively.

Also, because the conventional urethra catheter includes the balloon to fix the catheter inside of the bladder and consequently the inlet at the end of the inserted part should be apart from the entrance of the bladder, the urine can start to flow into the inlet of the inserted part after the level of the urine rises to a certain level. Thus, the possibility of infection increases due to the remaining urine in the bladder. However, according to the present invention, the expanding part that fixes the indwelling urinary catheter in the bladder acts as an inlet. In turn the inlet is positioned adjacent to the entrance of the bladder. Consequently, no remaining urine collects. Finally, the indwelling urinary catheter has a long life and can be manufactured economically and quickly due to the simple and effective structure.

While the present invention has been particularly shown and described with reference to the preferred embodiments thereof, it will be appreciated that many variations, modifications, and other applications of the present invention may be made without affecting the spirit and scope.

1. An indwelling urinary catheter for insertion into the bladder of the patient for urination, comprising:
a body that includes an inserted part for insertion into the bladder, and non-inserted part that is formed in one body with the inserted part and positioned outside of the bladder;
a discharging tube installed inside of the body for discharging urine;
a first fixing member formed inside of the inserted part; and
an expander, one end of which is fixed to the first fixing member.

2. The indwelling urinary catheter as in claim 1, wherein plural openings are formed in the inserted part adjacent to the non-inserted part.

3. The indwelling urinary catheter as in claim 2, wherein the openings have a shape selected from among an extended track shape, a lozenge shape with one side extended, an oval shape, and a polygon shape.

4. The indwelling urinary catheter as in claim 2, wherein the expander has a wire shape.

5. The indwelling urinary catheter as in claim 4, wherein the expander is made of one among piano wire, steel wire, or fishing-type line.

6. The indwelling urinary catheter as in claim 4, wherein a part of the inserted part expands in the lateral direction of the body with the openings as a center by pulling the expander.

7. The indwelling urinary catheter as in claim 6, wherein an indicator is further included in the expander to indicate when the inserted part has expanded.

8. The indwelling urinary catheter as in claim 7, wherein the indicator is an area or plural points having a different color in contrast to the entire expander.

9. The indwelling urinary catheter as in claim 6, wherein an inlet hole is formed in one end of the discharging tube positioned inside of the body, and an outlet hole is formed in the other end of the discharging tube positioned outside of the body.

10. The indwelling urinary catheter as in claim 9, wherein the second fixing member is formed in the other end of the discharging tube to fix the other end of the expander.

11. The indwelling urinary catheter as in claim 10, wherein the second fixing member is one selected from among a protrusion, a recess, or a combination of the protrusion and recess formed on the outer face of the discharging tube.

12. The indwelling urinary catheter as in claim 2, wherein the first fixing member has an opened-ring shape or a closed-ring shape.

13. The indwelling urinary catheter as in claim 2, wherein a contacting member is installed next to the openings of the inserted part.

14. The indwelling urinary catheter as in claim 13, wherein a ring shaped recess is formed between the inserted part and non-inserted part to affix the contacting member in the body.

15. The indwelling urinary catheter as in claim 1, wherein plural inlet holes are formed on the outer face of the one end of the inserted part in which the urine of the bladder flows.

16. The indwelling urinary catheter as in claim 2, wherein the one end of the discharging tube has a cone shape, center hole is formed in the center of the cone, plural inlet holes are formed in the circumference of the cone, and an outlet hole is formed in the other end of the discharging tube positioned outside of the body.

17. The indwelling urinary catheter as in claim 16, wherein a first thread is formed on the inner face of the non-inserted part, and a second thread corresponding to the first thread is formed on the outer face of the discharging thread.

18. The indwelling urinary catheter as in claim 17, wherein the second fixing member that is one selected from among a protrusion, a recess, or a combination of the protrusion and recess is formed in the other end of the discharging tube, and the one end of the expander is rolled and fixed to the second fixing member.

19. The indwelling urinary catheter as in claim 18, wherein as the discharging tube is withdrawn to the outside by rotation, consequently the one end of the expander is rolled to the second fixing member, and the inserted part expands partially in the lateral direction of the body with the opening as the center.

20. The indwelling urinary catheter as in claim 19, wherein an indicator is formed on the outer face of the other side of the discharging tube, by which the expanding state of the inserted part can be indicated.

21. The indwelling urinary catheter as in claim 2, wherein the one end of the expander is fixed in the one end of the discharging tube positioned inside of the body.

22. The indwelling urinary catheter as in claim 21, wherein the one end of the discharging tube has a cone shape, the expander is fixed in the center of the cone, plural inlet holes are formed in the circumference of the cone, and an outlet hole is formed in the other end of the discharging tube positioned outside of the body.

23. The indwelling urinary catheter as in claim 22, wherein a contacting member to prevent the urine from leaking is installed next to the inlet holes of the discharging tube.

24. The indwelling urinary catheter as in claim 23, wherein a membrane to prevent the urine from leaking is attached in the outlet hole of the discharging tube.

25. The indwelling urinary catheter as in claim 24, wherein an external fixing member is formed in the one end of the discharging tube.

26. The indwelling urinary catheter as in claim 25, wherein the one end of the external fixing member is fixed to the discharging tube, and the one end of the external fixing member received inside of the non-inserted part expands and is fixed to the one end of the non-inserted part when withdrawing the discharging tube.

27. The indwelling urinary catheter as in claim 26, wherein the external fixing member is a protrusion.

28. The indwelling urinary catheter as in claim 27, wherein the first recess to receive the protrusion and the second recess to fix the discharging tube when withdrawing the discharging tube are formed in one end of the non-inserted part, and the first recess and the second recess are formed at different depths.

29. The indwelling urinary catheter as in claim 1, wherein the one end of the non-inserted part is made of plastic.

30. An indwelling urinary catheter for insertion into the bladder of the patient for urination, compring:
a body that includes an inserted part to be inserted into the bladder, and non-inserted part that is formed in one body with the inserted part and positioned outside of the bladder;
an expanding part formed between the inserted part and non-inserted part;
a discharging tube installed inside of the body for discharging the urine installed;
a fixing member formed inside of the one end of the inserted part; and
an expander one end of which is fixed to the fixing member.

31. The indwelling urinary catheter as in claim 30, wherein the expanding part has a ring shape, on the outer face of which plural openings are formed.

32. The indwelling urinary catheter as in claim 31, wherein to prevent the expanding part from separating, the first protrusion that is inserted into the one end of the expanding part is formed on one end of the inserted part and the second protrusion that is inserted into the other end of the expanding part is formed on one end of the non-inserted part.
